(19) United States
(12) Patent Application Publication
(10) Pub. No.: US 2023/0012728 A1
(43) Pub. Date: Jan. 19, 2023

(54) TISSUE TRACTION DEVICES, SYSTEMS, AND METHODS

(71) Applicant: BOSTON SCIENTIFIC SCIMED, INC., MAPLE GROVE, MN (US)

(72) Inventors: Amanda Lynn Smith, Boston, MA (US); Paul Smith, Smithfield, RI (US); Kathryn Gildersleeve, Maple Grove, MN (US)

(73) Assignee: BOSTON SCIENTIFIC SCIMED, INC., MAPLE GROVE, MN (US)

(21) Appl. No.: 17/862,945

(22) Filed: Jul. 12, 2022

Related U.S. Application Data
(60) Provisional application No. 63/221,024, filed on Jul. 13, 2021.

(51) Int. Cl. A61B 17/02

(52) U.S. Cl. CPC A61B 17/0218; A61B 2017/00296

(57) ABSTRACT

A tissue traction device, system, and method presenting a grasping rail for coupling with a portion of a target tissue at a treatment site to apply a force, such as traction, to the grasped tissue, such as to facilitate performance of a procedure on the target tissue. A tissue traction device, system, and method having a distal support element, a proximal support element, and at least one expansion element extending therebetween. The at least one expansion element is extendable to define a working area for a procedure to be formed on target tissue at a treatment site. Two or more expansion elements may define a working area unimpeded by the expansion elements. The distal support element may be expandable to anchor the tissue traction device with respect to tissue.

### CROSS-REFERENCE TO RELATED APPLICATIONS

This application claims the benefit of priority of U.S. Provisional Application No. 63/221,024, filed Jul. 13, 2021, the entire disclosure of which is hereby incorporated by reference herein for all purposes.

### FIELD

The present disclosure relates generally to the field of devices, systems, and methods for applying traction to tissue.

### BACKGROUND

Various endoscopic surgical procedures require maneuvering about various anatomical structures. Some procedures, such as endoscopic mucosal resection (EMR), Endoscopic Submucosal Dissection (ESD), Pre-Oral Endoscopic Myotomy (POEM), etc., allow for minimally invasive endoscopic removal of benign and early malignant lesions, such as in the gastrointestinal (GI) tract. Because such procedures are minimally invasive, there is limited space to maneuver within the body. In procedures involving cutting of tissue, the loose section of tissue may obstruct visibility, such as by falling on the endoscope, occluding visibility of the camera, and creating a hindrance affecting movement of the instruments used during the procedure and in reaching the extreme corners of the target tissue being cut. Various solutions for lifting the hanging mass of tissue, thus clearing the path for visibility and operation of medical tools and devices, have been developed. However, positioning and maneuvering the elements used with such solutions may be challenging. Also, the elements used with such solutions may require separate medical tools than those used to perform the procedure, and such tools may even require a separate working channel in the endoscope, thereby potentially increasing the size and/or complexity of the endoscope. Alternative solutions for lifting tissue during a procedure which reduce cost, complexity, and cognitive load presented by currently available solutions would be welcome.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a tissue traction device includes a distal support element, a proximal support element, and a grasping rail extendable between the distal support element and the proximal support element. In accordance with an aspect of the present disclosure, the distal support element and the proximal support element are positionable spaced apart from one another on different sides of a target tissue at a treatment site with the grasping rail extended from the distal support element to the proximal support element, and the grasping rail is movable from an initial position spaced apart from the target tissue to engage the target tissue and to exert a force on the target tissue.

In some embodiments, the distal support element is expandable to be anchored with respect to tissue adjacent the treatment site.

In some embodiments, the grasping rail extends from a side of at least one of the distal support element or the proximal support element at an angle directed to another side of the at least one of the distal support element or the proximal support element.

In some embodiments, the force exerted by the grasping rail on the target tissue is in a direction away from the treatment site to hold the target tissue taut. Additionally or alternatively, in some embodiments, the grasping rail is movable proximally to exert a force on the grasped target tissue. Additionally or alternatively, in some embodiments, the grasping rail is formed of a shape memory material which exerts a force on the grasped target tissue.

In some embodiments, the tissue traction device includes a tissue grasper on the grasping rail configured to grasp the target tissue and to couple the grasping rail with the target tissue.

In some embodiments, the tissue traction device includes one or more expansion elements extending between the distal support element and the proximal support element, and extendable when the tissue traction device is positioned adjacent target tissue to define a working area around the treatment site.

In some embodiments, the tissue traction device includes one or more expansion elements extending between the distal support element and the proximal support element, and extendable when the tissue traction device is positioned adjacent target tissue to hold tissue surrounding the treatment site taut.

In accordance with various principles of the present disclosure, a tissue traction system includes a tissue traction device including a distal support element, a proximal support element, and a grasping rail extendable between the distal support element and the proximal support element; a delivery device with a lumen extending therethrough through which at least a component of the tissue traction device is deliverable to a treatment site, and a medical instrument deliverable by the delivery device. In accordance with an aspect of the present disclosure, the distal support element and the proximal support element are positionable spaced apart from each other on different sides of a target tissue at a treatment site with the grasping rail extended from the distal support element to the proximal support element, the grasping rail is movable from an initial position spaced apart from the target tissue to engage the target tissue and to exert a force on the target tissue, and the medical instrument is advanceable to the target tissue to perform a procedure on the target tissue.

In some embodiments, the grasping rail exerts a force on the target tissue in a direction away from the treatment site as the medical instrument is used to perform the procedure.

In some embodiments, the tissue traction device includes a tissue grasper configured to couple the grasping rail with the target tissue. In some embodiments, the tissue grasper is provided on the grasping rail and the grasping rail is moved towards the target tissue to cause the tissue grasper to grasp the target tissue and to couple the grasping rail with the target tissue. In some embodiments, the tissue grasper is advanced through the delivery device separately from the grasping rail and couples the grasping rail to the target tissue.

In some embodiments, the tissue traction device includes one or more expansion elements extending between the distal support element and the proximal support element, and extendable when the tissue traction device is positioned adjacent target tissue to define a working area in which an instrument may be advanced unobstructed by any of the expansion elements.

In accordance with various principles of the present disclosure, a method of moving a region of target tissue away from a treatment site includes deploying a tissue traction device having a proximal support element, a distal support element, and a grasping rail adjacent the treatment site with the grasping rail spaced apart and across from the treatment site; engaging the grasping rail with the target tissue, and moving the grasping rail away from the treatment site to move the grasped target tissue away from the treatment site.

In some embodiments, the method includes performing a procedure on the target tissue.

In some embodiments, the method including continuously applying force, via the grasping rail, on the grasped target tissue in a direction away from the treatment site to maintain the target tissue taut for performing the procedure thereon.

In some embodiments, the method includes extending one or more expansion elements of the tissue traction device against tissue around the treatment site to form a working area for performing the procedure on the target tissue.

In some embodiments, the method includes extending one or more expansion elements of the tissue traction device against tissue around the treatment site to hold the tissue of the treatment site taut.

In accordance with another aspect of the present disclosure, a tissue traction device includes a distal support element and a proximal support element and at least one expansion element extending from the distal support to the proximal support. In some embodiments, at least one of the a distal support element or a proximal support element is expandable to be anchored with respect to tissue adjacent the treatment site. The at least one expansion element is configured to move or to be moved from a delivery configuration, in which the at least one expansion element does not engage tissue, to a deployment configuration, in which the at least one expansion element moves or is moved radially or laterally outwardly, such as away from the longitudinal axis of the delivery device, to engage tissue at the treatment site. The at least one expansion element may engage the tissue to maintain tissue at the treatment site in a taut configuration to facilitate performance of a procedure at the treatment site. The at least one expansion element may be arranged to define a working space or working area in which to perform a procedure on the target tissue without obstructing instruments used to perform the procedure. In some embodiments, the tissue traction device is deployed in a body lumen and at least two expansion elements extend outwardly to engage tissue to define a working space. In some embodiments, the at least two expansion elements define a working space which is at least a semicircular area free of obstruction or interference by an expansion element (without the expansion elements extending within the working area). In some embodiments, the expansion elements hold tissue at the treatment site taut without extending into the working area.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of a tissue traction device in accordance with various aspects of the present disclosure.

FIG. 2 illustrates a perspective view of an embodiment of a tissue traction device in accordance with various aspect of the present disclosure in a delivery configuration.

FIG. 3 illustrates a perspective view of an example of a deployed traction section which may be provided in conjunction with a tissue traction device such as illustrated in FIG. 2.

FIG. 4 illustrates a perspective view of another embodiment of a tissue traction device in accordance with various aspect of the present disclosure.

FIGS. 5A-5G illustrate elevational views of various stages of use of a tissue traction device in accordance with various principles of the present disclosure.

FIG. 6 illustrates an alternate configuration for a tissue engaging element which may be used in the tissue traction device illustrated in FIGS. 5A-5G.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery). “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore.

In accordance with various principles of the present disclosure, a tissue traction system includes a tissue traction device, a delivery device, and optionally one or more medical instruments. A control handle may be provided along the proximal end of the delivery device and/or the tissue traction device and/or other instruments delivered or associated with the tissue traction device to facilitate control of the delivery device and/or the tissue traction device proximal to, and generally outside, the patient's body. It will be appreciated that terms such as control, move, manipulate, maneuver, steer, navigate, etc. (and conjugations and other grammatical forms thereof) may be used interchangeably herein without intent to limit.

The delivery device may be in the form of a flexible elongate member with one or more delivery lumens/working channels defined therein and extending substantially longitudinally (axially) between the proximal end and the distal end of the delivery device. It will be appreciated that the term flexible elongate member is used herein for the sake of convenience as an example of a delivery device in general and without intent to limit, and may be in the form of a catheter, sheath, tube, cannula, etc. (such terms being used interchangeably herein without intent to limit) or other configuration of an introducer. The delivery device is configured to deliver a tissue traction device, such as along a distal end thereof, or carried by another device, such as an endoscope. Additional elements, such as medical instruments, tissue graspers, clips, etc. may also be delivered by the delivery device to the treatment site. The delivery device may be any suitable size, cross-sectional shape or area, and/or configuration permitting introduction and passage of devices or instruments to the distal end of the delivery device. Use of a delivery device may advantageous to protect against adverse interactions of the tissue traction device and/or tissue traction system with (e.g., edges of the tissue retraction system catching, scratching, and/or otherwise) other elements such as the passage (body passage or working channel of another device) through which the delivery device is advanced. Additional overtubes or sheaths may be provided as desired or as necessary to reduce friction or interference of the exterior of any elements with another element. It is generally beneficial for the delivery device to be steerable, and the delivery device may have different areas of different flexibility or stiffness to promote steerability.

The delivery device and/or overtube(s) associated therewith may be made from any suitable biocompatible material known to one of ordinary skill in the art and having sufficient flexibility to traverse non-straight or tortuous anatomy. Such materials include, but are not limited to, rubber, silicon, synthetic plastic, stainless steel, metal-polymer composite; metal alloys of nickel, titanium, copper cobalt, vanadium, chromium, and iron; superelastic or shape memory material such as nitinol (nickel-titanium alloy) or a spring steel; different layers of different materials and reinforcements. Such materials may be made of or coated with a polymeric or lubricious material to enable or facilitate passage of a deliver device therethrough. In some embodiments, the working channels may be made of or coated with a polymeric or lubricious material to facilitate passage of the introduced medical instrument(s) through the working channel(s). Reference may be made herein to a medical instrument or tool or device (such terms as instrument, tool, device, etc. being used interchangeably herein and without intent to limit), such as, without limitation, grasper, forceps, snare, scissors, knife, dissector, clamp, endoscopic stapler, tissue loop, clip applier, suture-delivering instrument, energy-based tissue coagulator or cutter, etc. Such instruments may be used to perform a procedure or operation which is either diagnostic or therapeutic or both, such as grasping, resecting, dissecting, retracting, cutting, and/or otherwise manipulating tissue. It will be appreciated that the particular instrument or procedure performed therewith is not critical to the present disclosure. The medical instrument may also include visualization and/or imaging devices, including, without limitation, various endoscopes known in the art.

A tissue traction device formed in accordance with various principles of the present disclosure may be used to lift target tissue (a designated section of tissue) away from a target tissue area at which a procedure is being performed. In some instances, the target tissue is an unhealthy, diseased (i.e., cancerous, pre-cancerous etc.), or otherwise undesirable portion of tissue that may be healthy or unhealthy. A “target tissue” may also include tissues that are suspected of being unhealthy or diseased, but which require surgical removal for verification of their disease status by biopsy. It should be appreciated that surgical dissection or resection of a target tissue typically includes removal of a portion of the surrounding healthy tissue along the target tissue margin to ensure complete removal and to minimize the potential for metastasis of left-behind or dislodged target tissue cells to other body locations. The target tissue is within a target tissue area in the body, such as the gastrointestinal system. Terms such target tissue area, target tissue site, target area of tissue, target area, target site, target treatment area, treatment area, target treatment site, treatment site, etc., may be used interchangeably herein, without intent to limit, to refer to an area or region of tissue on which a procedure is to be performed or which is to be treated or otherwise operated on or affected by the devices and/or systems and/or methods disclosed herein, including areas or regions extending outwardly from or around or surrounding the target tissue (specific tissue in the target tissue area). Devices, systems, and methods disclosed herein may be used for treating body tissues, such as in the gastrointestinal system, the abdominal cavity, digestive system, urinary tract, reproductive tract, respiratory system, cardiovascular system, circulatory system, etc., without limitation. It will be appreciated that devices and systems disclosed herein are scalable to suit the intended use and/or treatment site. For instance, a tissue traction device may have an outer transverse dimension of approximately 1″-1.5″ (2.54 cm-3.81 cm) to be positioned in an esophagus; or approximately 3″ (7.62 cm) to be positioned in a stomach; or approximately 2″ (5.08 cm) to be positioned in a colon.

In accordance with various principles of the present disclosure, a tissue traction device is positioned at a treatment site to apply traction to the target tissue. It will be appreciated that reference is made herein to locations such as around, adjacent, etc., such terms (and other such terms) being used interchangeably herein without intent to limit to a confined location as the specific location may depend or vary based on the target tissue and/or condition being treated as may be determined by the medical professional. In some embodiments, a tissue traction device formed in accordance with various principles of the present disclosure may include a tissue grasping rail configured to be coupled with the target tissue and to apply traction to the target tissue. It will be appreciated that terms such as apply traction, withdraw, retract, pull, etc. (and conjugations and other grammatical forms thereof) may be used interchangeably herein without intent to limit, such as in describing forces on or movements of an element such as a target tissue. The tissue grasping rail (such term being used for the sake of convenience and without intent to limit) may be an element such as a rail or wire or band or cord or the like capable of being coupled to the target tissue and to apply a force or force vector or traction to the target tissue (all terms recited in the alternative being used interchangeably herein without intent to limit). For instance, if a procedure is being performed on the target tissue (such as with a medical instrument), it may be desirable to lift or to retract the tissue out of the field of vision or out of the way of the instrument being used to perform the procedure.

The grasping rail need not be rigid, and may generally be flexible and/or elastic to impart the desired force on the tissue coupled thereto. The grasping rail may be elastic or resilient or otherwise return to a rest or neutral or initial position (prior to being coupled to the target tissue) to exert traction force on the target tissue without application of an external force thereto. Additionally or alternatively, the grasping rail may be moved to engage the tissue and to retract the tissue (such as by a proximally directed force exerted by a medical professional at a proximal end of the grasping rail). The grasping rail may be configured with a tissue grasper (e.g., a hook, barb, clip, or other element configured to engage and to remain engaged with tissue) either integrally formed with (e.g., as a part of, and optionally from the material and/or structure of) the grasping rail or separately formed and coupled with the grasping rail. The tissue grasper is configured to grasp or to be coupled with tissue so that the grasping rail may impart a traction force to the tissue. A separate tissue grasper, such as a tissue clip, may be delivered separately from the grasping rail, coupled with the grasping rail, and extended towards tissue to be grasped to couple the grasping rail with such tissue. The grasping rail may include a tissue grasper engagement element such as a loop or ring or other structure facilitating grasping of the grasping rail by a tissue grasper. The tissue grasper engagement element may be integrally formed with (e.g., as a part of, such as from the material of) the grasping rail or may be separately formed and coupled thereto. In some embodiments, a tether (elongate member such as a traction band, elastic or rubber band, stretchable elongate element, wire, cord, cable, spring, suture, high carbon spring wire, nitinol, spring steel, music wire, muscle wire, and/or any other suitable elongate member) may be coupled between the grasping rail and the target tissue. The tether may be directly coupled to the grasping rail or coupled to the grasping rail with a clip or the like. A tissue grasper may be used to couple the tether with the target tissue.

A tissue traction device formed in accordance with various principles of the present disclosure may be configured to be delivered within a body passage or body lumen (e.g., intestines) or within an organ (e.g., stomach) presenting tissue walls forming an environment (lumen or cavity or other shape with the tissue wall curving to form a space therebetween) in which the tissue traction device may be seated and anchored against. The tissue walls need not be opposite one another, but may at least provide a sufficiently enclosed area with walls against which a portion of the tissue traction device may be securely positioned so as not to shift or move, such as tissue walls which may be engaged on either side of the target tissue. The treatment site need not have an upper wall (e.g., as in a tubular body passage with a tubular lumen therethrough), so long as the tissue traction device can hold sufficient traction with respect to the treatment site, as determinable by one of ordinary skill in the art. In some embodiments, the tissue traction device includes at least one support element configured to secure the tissue traction device in place with respect to the treatment site. For instance, if the tissue traction device is to be placed within a body lumen, the support element may be a generally rounded (e.g., circular, though not necessarily circular) structural element sized, shaped, and configured to engage the walls of the body lumen. The support element may be considered to secure, anchor, hold, or otherwise maintain the position of the tissue traction device relative to the treatment site. The at least one support element may be at least a distal support element positioned at a distal end of the tissue traction device with the grasping rail extending proximally therefrom. In some embodiments, at least the distal support element is an expandable element, and may be in the general configuration of an expandable ring. The expandable distal support element may be maintained in a compact delivery configuration and expanded (e.g., upon application of an expanding force thereto, or self-expanded without external forces) upon delivery to the treatment site. In an expanded configuration, the expandable distal support element expands to a dimension at least the same as, and optionally at least slightly larger than, a corresponding dimension (e.g., in substantially the same direction or shape or configuration) of the treatment site to maintain the distal support element in place relative to the treatment site. One of ordinary skill in the art can readily ascertain a “slightly larger” dimension sufficient to secure the expandable support element with respect to the target tissue without damaging or otherwise adversely impacting the treatment site.

A proximal support element may be provided along a proximal end of the grasping rail. The proximal support element may be a cap configured to be delivered over a distal end of a delivery device or directly over a distal end of an endoscope. Such proximal support element may be deployable from the element on which it is delivered to be seated along a proximal side of the treatment site. The proximal support element may be a distal portion or section of the delivery device. The proximal support element need not be sized to engage tissue wall (particularly if sized to be passed through a body lumen to the treatment site).

In some embodiments, the tissue traction device is released from the delivery device. In other words, the distal support element and the proximal support element are separated from the delivery device and deployed at the treatment site. In some embodiments, the distal support element and the proximal support element remain coupled to the delivery device.

Additionally or alternatively, the tissue traction device may include one or more expansion elements configured to engage tissue surrounding the treatment site to hold tissue at the treatment site in sufficient tension (e.g., sufficiently taut) to facilitate performance of the desired/necessary procedure on the target tissue. In contrast with a cage type expansion structure with multiple expansion elements, in accordance with various principles of the present disclosure, the one or more expansion element are extendable to define a working area around the target tissue which is clear of expansion elements. In other words, the expansion elements are positioned to define a working area and to hold tissue taut without extending into the working area. For instance, the expansion elements may define an approximately 180° portion of a generally circular body site (e.g., a body lumen) as a working area with an expansion element on either side thereof without extending into such working area. Two expansion elements may define an area to one side of a plane in which the two expansions elements lie (each in contact with tissue surrounding the treatment site) and of sufficient height to allow manipulation and the appropriate freedom of motion of the instrument being used on the target tissue. In some embodiments, only two expansion elements are provided to maximize the working area unimpeded by the expansion elements while allowing the expansion elements to maintain the tissue in the region of the target tissue sufficiently taut as desired to facilitate performance of a procedure on or treatment of (e.g., cutting, dissection, resection, etc., such terms being used interchangeably herein without intent to limit) the target tissue. The expansion elements may be a wire or rail or other elongated element capable of exerting a force on tissue around a treatment site. The expansion elements may be flexible and may shift from a delivery configuration to an expanded deployment configuration. In the deployment configuration, the expansion elements may extend laterally or radially away, e.g., outwardly away, from the tissue traction device (such as away from a longitudinal axis, such as a central axis, of the tissue traction device). In some embodiments, the expansion elements are self-expanding, or are otherwise formed to extend on their own and without external force applied thereto once deployed. For instance, the expansion elements may be formed of a shape memory material (e.g., nitinol) or a spring steel shaped and configured to expand or otherwise form a cage or scaffold or the like once deployed, such as to create a working area.

An overtube or other sheath may be provided with a lumen therethrough for guiding the grasping rail and/or the expansion elements. The element from which the grasping rail and/or the expansion elements extend to the treatment site may be configured to direct the grasping rail and/or the expansion elements in a selected direction to facilitate and/or enhance the performance of the grasping rail and/or the expansion elements. For instance, the grasping rail may be directed towards an opposite side of the tissue traction device and/or radially inwardly such that when positioned in the region of the treatment site, the grasping rail is extended towards the target tissue. Additionally or alternatively, the expansion elements may be directed laterally or radially outwardly in a general direction in which the expansion elements are to be extended.

The grasping rail and/or the expansion elements may extend proximally through or along the delivery device, such as to a control handle configured to control movement (e.g., distal advancement or proximal retraction) of the grasping rail and/or expansion elements. The control handle may be used to adjust the position of the grasping rail to engage with the target tissue and/or to cause the expansion elements to extend/expand to hold tissue at the treatment site in tension and/or to control other elements of the tissue traction system or associated therewith.

Various embodiments of a tissue traction device and system and associated methods will now be described with reference to examples illustrated in the accompanying drawings. Reference in this specification to “one embodiment,” “an embodiment,” “some embodiments”, “other embodiments”, etc., indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. It should be understood that one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Therefore, the present disclosure is not limited to only the embodiments specifically described herein.

Turning now to the drawings, it will be appreciated that in the following description, elements or components similar among the various illustrated embodiments are generally designated with the same reference numbers increased by 100 and redundant description is omitted. Common features are identified by common reference elements and, for the sake of brevity, the descriptions of the common features are generally not repeated, reference being made to the prior descriptions of similar elements and operations. Certain features in one embodiment may be used across different embodiments and are not necessarily individually labeled when appearing in different embodiments. For purposes of clarity, not all components having the same reference number are numbered.

An example of an embodiment of a tissue traction device 100 formed in accordance with various principles of the present disclosure is illustrated in FIG. 1 in a deployed configuration with a distal support element 110 (at a distal end 101 of the tissue traction device 100) spaced apart (distally) from a proximal support element 120 (at a proximal end 103 of the tissue traction device 100). In the deployed configuration illustrated in FIG. 1, a grasping rail 130 extends between the proximal support element 120 and the distal support element 110 in a neutral, initial position ready to engage target tissue. In the illustrated embodiment, a tissue grasper 132 is provided or formed on the grasping rail 130. It will be appreciated that a separately formed tissue grasper 132 or no tissue grasper 132 may be provided on the grasping rail 130. One or more expansion elements 140 also extend between the proximal support element 120 and the distal support element 110. As discussed above, the tissue traction device 100 may be deployed as part of a tissue traction system which may also include a delivery device and one or more medical instruments. A tissue traction device formed in accordance with any of the various principles of the present disclosure may be delivered in any of a variety of manners with or by the delivery device, optionally in conjunction with at least one medical instrument. The present disclosure encompasses the use of introducers, such as a delivery sheath, in a variety of different forms, such as with an overtube forming a proximal support element or with a cap over an endoscope forming a proximal support element.

The distal support element 110 may be delivered to a treatment site spaced apart from the proximal support element 120 or in a position adjacent the proximal support element 120. The distal support element 110 of the tissue traction device 100 may be delivered mounted on a portion of the tissue traction system, such as on the proximal support element 120, or on delivery device with which the tissue traction device 100 is delivered. For instance, a recess or other holding structure may be provided for engagement by the distal support element 110 in a delivery configuration. In some embodiments, the distal support element 110 is expandable, and held in a generally contracted delivery configuration during delivery to the treatment site.

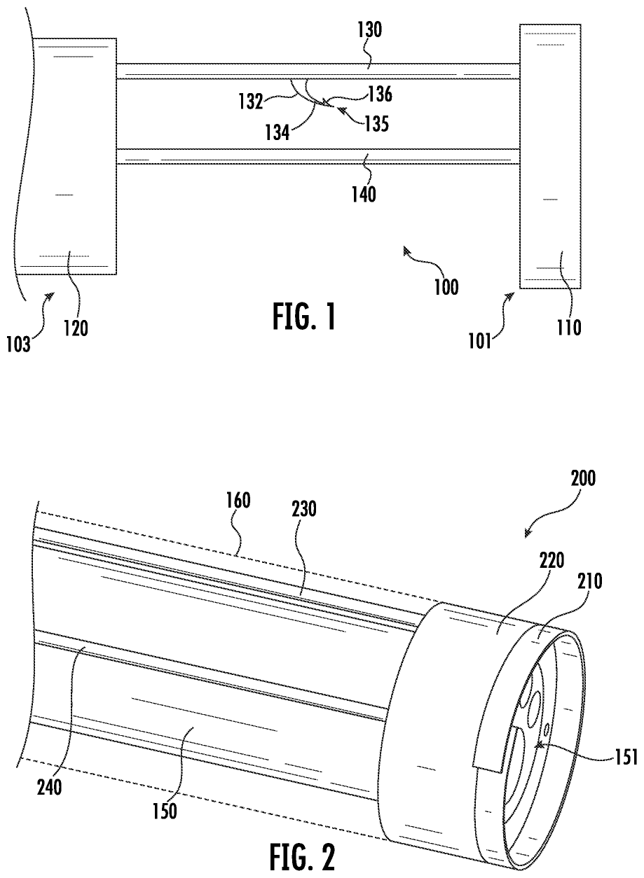

An example of an embodiment of a tissue traction device 200 is illustrated in FIG. 2 in an example of a delivery configuration with a distal support element 210 delivered adjacent proximal support element 220. The proximal support element 220 is in the form of a cap configured to be placed over an endoscope 150, such as over a distal end 151 of an endoscope 150 (which may be considered a part of the tissue traction system). A recess or other holding structure may be provided in the proximal support element 220 or on the delivery device or on the endoscope 150 for engagement by the distal support element 210 in a delivery configuration. Optionally, the tissue traction device 200 and the endoscope 150 may be delivered via a delivery device 160 such as a flexible elongate member, delivery catheter, overtube, sheath, etc., which may be considered a part of the tissue traction system as well.

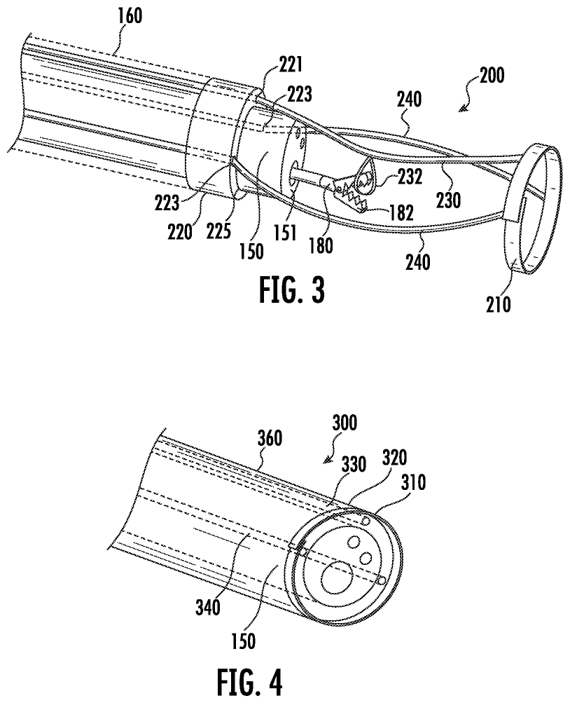

In a deployment configuration, such as illustrated in FIG. 3, the distal support element 210 may be extended distally away from the proximal support element 220. In some embodiments, the distal support element 210 is delivered in a contracted delivery configuration and optionally expanded into a deployment configuration to engage tissue in the region of a treatment site TS. A grasping rail 230 and expansion elements 240 extend between the proximal support element 220 and the distal support element 210. Lumens 221, 223 may be defined in the proximal support element 220 (as may be seen in FIG. 3) to allow the grasping rail 230 and the expansion elements 240 to move relative to the proximal support element 220 to allow the proximal support element 220 and the distal support element 210 to be positioned closer together for delivery, as in FIG. 2, than in the deployed configuration illustrated in FIG. 3. The grasping rail 230 and expansion elements 240 may extend proximally to a proximal end of the tissue traction device 200 and/or the tissue traction system, such as to a control handle or the like. The delivery device 160 may protect or otherwise shield the grasping rail 230 and/or the expansion elements 240. For instance, guide lumens may be defined in the delivery device 160 for passage of the grasping rail 230 and/or the expansion elements 240 therethrough. Alternatively, the grasping rail 230 and/or the expansion elements 240 may be provided with individual sheaths (such as Bowden cables) to guide movement thereof and to protect the grasping rail 230 and expansion elements 240 and body passages through which the grasping rail 230 and expansion elements 240 extend. Alternatively, the grasping rail 230 and the expansion elements 240 may be slidable with respect to the proximal support element 220 to allow relative movement between the proximal support element 220 and the distal support element 210 (e.g., to change the distance therebetween), and proximal ends of the grasping rail 230 and/or the expansion elements 240 may be provided with widened portions or stop elements preventing detachment or exiting of the grasping rail 230 and the expansion elements 240 from the respective lumens 221, 223 in the proximal support element 220.

In another example of an embodiment of a tissue traction device 300, illustrated in FIG. 4, the proximal support element 420 may be a distal portion of an overtube 360 which may serve as a delivery device 360 for the tissue traction device 300 and other components of the tissue traction system, such as an endoscope 150. As may be appreciated with reference to FIG. 4, the distal support element 310 may be mounted on a portion of the tissue traction system, such as on the distal end of the delivery device 360 forming the proximal support element 320. For instance, a recess or other holding structure may be provided for engagement by the distal support element 310 in a delivery configuration. In some embodiments, the distal support element 310 is expandable, and held in a generally contracted delivery configuration during delivery to the treatment site, such as illustrated in FIG. 4. The distal support element 310 may be moved distally away from the proximal support element 320 in a deployed configuration similar to the configuration illustrated in FIG. 3, with a grasping rail 330 and expansion elements 340 (illustrated in broken lines within the delivery device 360) extended between the proximal support element 320 and the distal support element 310. In some embodiments, guide lumens are defined in the delivery device 360 for passage of the grasping rail 330 and/or expansion elements 340 therethrough, such as to a proximal location outside the body to allow a medical professional to control movement of the grasping rail 330 and/or expansion elements 340, such as in a manner described herein. A control handle may be provided, such as noted above.

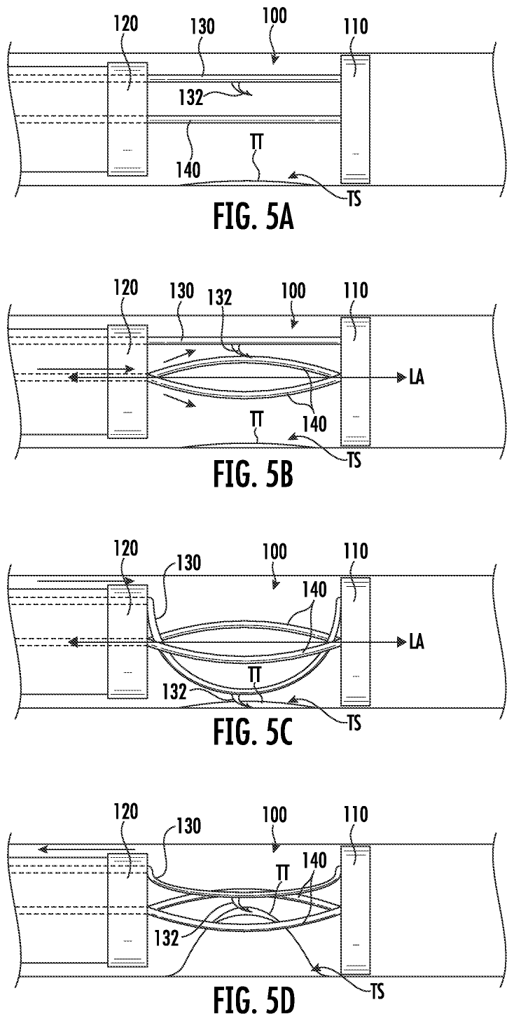

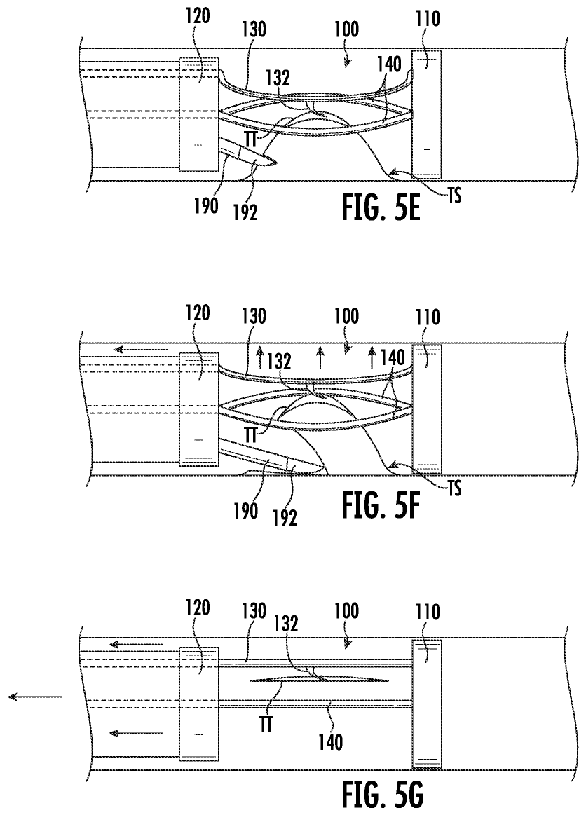

Once the tissue traction device 100, 200, 300 is deployed, the grasping rail 130, 230, 330 thereof may be used to apply a force to the target tissue on which a procedure is to be performed. An example of an embodiment of a tissue traction device 100 and use of the grasping rail 130 thereof to apply a force to target tissue TT is illustrated in FIGS. 5A-5G. For the sake of convenience and simplicity, and without intent to limit, reference is made herein generally to reference numbers used in the embodiment illustrated in FIG. 1. However, it will be appreciated that any of the other embodiments of tissue traction devices described herein or in accordance with various principles of the present disclosure described herein, such as illustrated in the accompanying drawings or formed in accordance with various principles of the present disclosure, may be used instead, the manner of use not being limited by specific configurations of components or arrangements of a tissue traction device formed in accordance with various principles of the present disclosure.

An example of an embodiment of a tissue traction device 100 is illustrated in FIG. 5A in a deployed configuration in the region of a treatment site TS. The distal support element 110 of the tissue traction device 100 is positioned in a deployed configuration (e.g., expanded to contact tissue walls at the treatment site TS, such as to extend about and to engage the walls of a body lumen) distal to a target tissue TT. The proximal support element 120 of the tissue traction device 100 is positioned proximal to the target tissue TT. Accordingly, the grasping rail 130, extending between the distal support element 110 and the proximal support element 120, is positioned in the region of the target tissue TT (e.g., opposite, spaced apart, above, alongside, etc.) to facilitate being coupled thereto to exert a force on the target tissue TT. For the sake of convenience, the target tissue TT will be described as being along a bottom or lower region of the anatomical structure in/on/along which the treatment site TS is located, and references herein are made with respect to such orientation, although any orientation is feasible as may be appreciated by one of ordinary skill in the art. As such, the grasping rail 130 is illustrated as being spaced apart from the target tissue TT with the tissue grasper 132 thereof positioned above the target tissue TT, but other orientations are within the scope and spirit of the present disclosure with similar relative positions between the target tissue TT and the grasping rail 130 and tissue grasper 132 thereof. Generally, the grasping rail 130 may be considered to extend from one side of the tissue traction device 100 towards another side (e.g., opposite side) of the tissue traction device 100 to be engaged with or coupled to the target tissue TT.

Once the proximal support element 120 and the distal support element 110 have been positioned as desired (with the grasping rail 130 in position to be engaged with the target tissue TT, such as with the tissue grasper 132), the expansion elements 140 may be extended/expanded to define a working area in which the target tissue TT may be accessed and/or a procedure performed thereon, as illustrated in FIG. 5B. The expansion elements 140 may be manipulated to be extended outwardly to engage tissue walls along the sides or otherwise adjacent or surrounding the treatment site TS. More particularly, the expansion elements 140 may be extended radially or laterally outward (e.g., to bow or flex) away from a longitudinal axis LA of the tissue traction device 100 to engage tissue. Such engagement of tissue in the area of the treatment site TS may apply a force on the tissue to hold the tissue taut to facilitate performance of a procedure thereon, such as on the target tissue TT. In some embodiments, the expansion elements 140 are coupled to or extend from the proximal support element 120 at an angle. For instance, the guide lumens through the proximal support element 120 through which the expansion elements 140 extend (see, e.g., guide lumens 223 illustrated in FIG. 3) may be at an angle relative to the longitudinal axis LA of the tissue traction device 100 so that the expansion elements 140 extend outwardly from the proximal support element 120 with respect to the longitudinal axis LA (e.g., a central longitudinal axis LA). In some embodiments, the guide lumens and the expansion elements 140 may be keyed to inhibit or prevent rotational flipping of the expansion elements 140 to maintain the desired exit angle and direction of the expansion elements 140.

In some embodiments, as described above, the grasping rail 130 and/or the expansion elements 140 may extend proximally to a proximal location outside the body to allow a medical professional to control movement of the grasping rail 130 and/or the expansion elements 140. In such embodiments, the expansion elements 140 may be pushed into an expanded configuration (e.g., by being pushed distally with respect to the proximal support element 120). A control handle may be provided along a proximal end of the tissue traction device 100 and/or the tissue traction system to control such movement of the expansion elements 140 and optionally any other element or elements of the tissue traction system. The configuration of such control handle may be any known or heretofore known configuration, the present disclosure not being limited to a particular configuration as such configuration is not critical to the general principles of the present disclosure.

In embodiments in which the expansion elements 140 do not extend proximally to a control handle (e.g., in embodiments in which the expansion elements 140 have proximal ends coupled to the proximal support element 120, or proximal ends or slidable with respect to yet held such as by widened areas against separation from the proximal support element 120), the expansion elements 140 may extend outwardly upon deployment of the tissue traction device 100. For instance, the expansion elements 140 may be formed of shape memory material such as Nitinol or a spring steel held in a substantially straight configuration (generally requiring the expansion elements 140 to be held within a sheath, such as within the delivery device 160, with the proximal support element 120 and the distal support element 110 delivered within the sheath). Once deployed, and any retention force or other device holding the expansion elements 140 in a delivery configuration (e.g., unexpanded configuration) is removed, the expansion elements 140 may move independently of an external force into a predetermined expanded configuration as desired, in a manner known or heretofore known to those in the art (the details of which are not critical to the principles of the present disclosure).

In some embodiments, the working area defined by the expansion elements 140 allows sufficient space for a introduction of a medical instrument to be used to perform a procedure on the target tissue TT without interference from the expansion elements 140. In other words, the expansion elements 140 may be spaced apart to surround, without extending into, the working area. If the treatment site TS is a body lumen, the expansion elements 140 may be positioned substantially midway across the distance from the target tissue TT to the opposite wall of the body lumen, leaving approximately half the circumferential area/surface of the body lumen in which the target tissue TT is located clear of components of the tissue traction device 100. In other words, the expansion elements 140 may extend a distance of at least half the diameter of the body lumen taken from the target tissue TT to the tissue wall opposite the target tissue TT. Such configuration affords a larger working area than generally provided by prior art tissue expansion systems.

With the tissue traction device 100 deployed, and optionally with the expansion elements 140 hold tissue in the treatment site TS taut, the grasping rail 130 may be engaged with the target tissue TT. In the example of an embodiment illustrated in FIG. 5C, the grasping rail 130 includes a tissue grasper 132 with a distal end 135 configured to engage and grasp (e.g., hold or be coupled to) tissue. Movement of the grasping rail 130 closer to the target tissue TT allows the tissue grasper 132 to engage the target tissue TT to be coupled therewith. Such movement of the grasping rail 130 may be achieved by controlling movement of the grasping rail 130 by moving a proximal end thereof positioned outside the body such as described with respect to the expansion elements 140. For instance, the grasping rail 130 may be pushed distally with respect to the proximal support element 120 to bow or flex towards the target tissue TT. In some embodiments, the grasping rail 130 is coupled to or extends from the proximal support element 120 at an angle. For instance, the guide lumen through the proximal support element 120 through which the grasping rail 130 extends (see, e.g., guide lumen 221 illustrated in FIG. 3) may be at an angle relative to the longitudinal axis LA of the tissue traction device 100 so that the grasping rail 130 extends towards an opposite side of the tissue traction device 100 (e.g., radially inwardly from the proximal support element 120 with respect to the longitudinal axis LA) and towards the target tissue TT. In some embodiments, the guide lumens and the grasping rail 130 may be keyed to inhibit or prevent rotational flipping of the grasping rail 130 to maintain the desired exit angle and direction of the grasping rail 130.

Once the tissue grasper 132 is engaged with the target tissue TT (e.g., as in FIG. 5C), the grasping rail 130 may be moved away from the target tissue TT to apply force/traction on the target tissue TT, such as illustrated in FIG. 5D. In the illustrated embodiment, which is similar to the embodiment illustrated in FIG. 1, the tissue grasper 132 includes a distally directed hook 134 with a barb 136 (proximally directed) adjacent the distal free end 135 of the tissue grasper 132. Such configuration allows the tissue grasper 132 to engage the target tissue TT and to be held thereto by the barb 136 so that movement of the grasping rail 130 away from the target tissue TT (see, e.g., FIG. 5D relative to FIG. 5C), such as by pulling the grasping rail 130 proximally, does not cause the tissue grasper 132 to disengage from the target tissue TT.

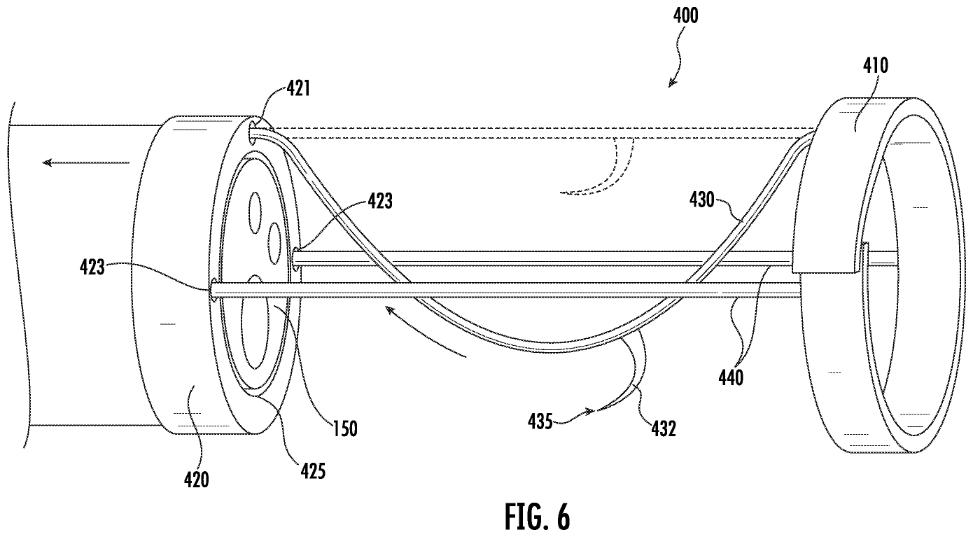

It will be appreciated that the tissue grasper may be in any other configuration suitable for grasping or being coupled with tissue such as the target tissue. For instance, in the example of an embodiment of a tissue traction device 400 illustrated in FIG. 6, a hook-like tissue grasper 432 similar to the tissue grasper 132 illustrated in FIG. 1 and FIGS. 5A-5G, but with a different orientation, may be provided. More specifically, the free end 435 of the tissue grasper 432 illustrated in FIG. 6 extends proximally, whereas the free end 135 of the tissue grasper 132 illustrated in FIG. 1 and FIGS. 5A-5G extends distally. The configuration of the tissue grasper 432 illustrated in FIG. 6 may enhance the hold of the tissue grasper 432 on the target tissue TT as the grasping rail 430 is moved away from the target tissue TT, particularly if the grasping rail 430 is secured to the distal support element 410 and pulled proximally through a guide lumen 421 defined in the proximal support element 420, to cause the tissue grasper 432 to hook more securely into the target tissue TT.

Alternatively, the grasping rail 130 may include a tissue grasper engagement element sized, shaped, and configured for being grasped by a separately formed tissue grasper such as a clip. An example of an embodiment of such configuration is illustrated in FIG. 3. An instrument 180 (e.g., grasper tool) carrying a tissue grasper 182 may be extended distally from the delivery device to the tissue traction device 200. For instance, the instrument 180 may be distally advanced through a working channel 151 of an endoscope 150 (which, in turn, may be advanced through a lumen 225 of the proximal support element 220 and a corresponding lumen through the delivery device 160 in communication with the lumen 225) The tissue grasper 182 is moved towards the tissue grasper engagement element 232 to grasp the tissue grasper engagement element 232. The tissue grasper 182 may then grasp the target tissue TT as well. The grasping rail 230 may be moved towards the target tissue TT to allow the tissue grasper 182 to reach the target tissue TT and/or the grasping rail 230 may be stretched or otherwise extended (such as by expanding an elastic/expandable grasping rail 230). The tissue grasper engagement element 232 may be in any desired shape or form or configuration, allowing grasping thereof by a tissue grasper 182 such as a clip. In the example of an embodiment illustrated in FIG. 3, the tissue grasper engagement element 232 is in the form of a loop. However, other suitable structures may be used as may be appreciated by those of ordinary skill in the art. For instance, a tether (such as an elastic tether) may be used to couple the grasping rail 230 and the target tissue TT together.

Once the target tissue TT has been adequately engaged with the grasping rail 130 (and optionally pulled into a taut configuration), such as illustrated in FIG. 5D, a medical instrument 190 may be advanced towards the working area, as illustrated in FIG. 5E. The medical instrument 190 may be advanced through a working channel of the endoscope and/or delivery device or in any manner known or heretofore known in the art. The medical instrument 190 may include a working end 192 configured to perform a procedure on the target tissue TT. For instance, the working end 192 of the medical instrument 190 may be a cutting end (e.g., blade, cauterization tip, etc.).

The grasping rail 130 continues to exert traction force on the target tissue TT as the indicated procedure is performed, such as with the medical instrument 190. The traction force may be as a result of continuous proximal movement of the proximal end of the grasping rail 130 (e.g., such as by a medical professional pulling proximally on the grasping rail 130) or as a result of a restoring force which restores an extended or flexed or elastically expanded grasping rail 130 back to its neutral position (e.g., a position as illustrated in FIG. 5C). In the example of an embodiment illustrated in FIG. 5F, in which the medical instrument 190 is a cutting instrument, as the target tissue TT is cut, the grasping rail 130 continues to exert traction on the target tissue TT to move the cut portion (e.g., flap) of the target tissue TT away from the medical instrument 190. Such use of the grasping rail 130 maintains a clear view of the treatment site TS, such as with the use of an endoscope, without the cut tissue creating a tunneling effect or otherwise obstructing the view.

Once the target tissue TT has been resected or dissected or otherwise cut away from the treatment site TS, such as illustrated in FIG. 5G, the tissue traction device 100 may be removed (e.g., proximally withdrawn) from the treatment site TS. The target tissue TT may remain couple with the grasping rail 130 and removed with the grasping rail 130 and the tissue traction device 100.

In view of the above, it should be understood that the various embodiments illustrated in the figures have several separate and independent features, which each, at least alone, has unique benefits which may be desirable for, yet not critical to, the presently disclosed tissue traction device, system, and method. Therefore, the various separate features described herein need not all be present in order to achieve at least some of the desired characteristics and/or benefits described herein. Only one of the various features may be present in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure.

It will further be appreciated that all apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used herein, the term “or” is generally employed in its sense including “and/or” unless the content clearly dictates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue traction device comprising:
a distal support element;
a proximal support element; and
a grasping rail extendable between said distal support element and said proximal support element;
wherein:
said distal support element and said proximal support element are positionable spaced apart from one another on different sides of a target tissue at a treatment site with said grasping rail extended from said distal support element to said proximal support element; and
said grasping rail is movable from an initial position spaced apart from the target tissue to engage the target tissue and to exert a force on the target tissue once engaged to the target tissue.

2. The tissue traction device of claim 1, wherein said distal support element is expandable to be anchored with respect to tissue adjacent the treatment site.

3. The tissue traction device of claim 1, wherein said grasping rail extends from a side of at least one of said distal support element or said proximal support element at an angle directed to another side of said at least one of said distal support element or said proximal support element.

4. The tissue traction device of claim 1, wherein the force exerted by said grasping rail on the target tissue is in a direction away from the treatment site to hold the target tissue taut.

5. The tissue traction device of claim 4, wherein said grasping rail is movable proximally to exert a force on the grasped target tissue.

6. The tissue traction device of claim 4, wherein said grasping rail is formed of a shape memory material which exerts a force on the grasped target tissue.

7. The tissue traction device of claim 1, further comprising a tissue grasper on said grasping rail configured to grasp the target tissue and to couple said grasping rail with the target tissue.

8. The tissue traction device of claim 1, further comprising one or more expansion elements extending between said distal support element and said proximal support element, and extendable when the tissue traction device is positioned adjacent target tissue to define a working area around the treatment site.

9. The tissue traction device of claim 1, further comprising one or more expansion elements extending between said distal support element and said proximal support element, and extendable when said tissue traction device is positioned adjacent target tissue to hold tissue surrounding the treatment site taut.

10. A tissue traction system comprising:
a tissue traction device comprising a distal support element, a proximal support element, and a grasping rail extendable between said distal support element and said proximal support element;
a delivery device with a lumen extending therethrough through which at least a component of said tissue traction device is deliverable to a treatment site; and
a medical instrument deliverable by said delivery device;
wherein:
said distal support element and said proximal support element are positionable spaced apart from each other on different sides of a target tissue at a treatment site with said grasping rail extended from said distal support element to said proximal support element;
said grasping rail is movable from an initial position spaced apart from the target tissue to engage the target tissue and to exert a force on the target tissue; and
said medical instrument is advanceable to the target tissue to perform a procedure on the target tissue.

11. The tissue traction device of claim 10, wherein said grasping rail exerts a force on the target tissue in a direction away from the treatment site as said medical instrument is used to perform the procedure.

12. The tissue traction device of claim 10, further comprising a tissue grasper configured to couple the grasping rail with the target tissue.

13. The tissue traction device of claim 12, wherein said tissue grasper is provided on said grasping rail and said grasping rail is moved towards the target tissue to cause said tissue grasper to grasp the target tissue and to couple the grasping rail with the target tissue.

14. The tissue traction device of claim 12, wherein said tissue grasper is advanced through said delivery device separately from said grasping rail and couples said grasping rail to the target tissue.

15. The tissue traction device of claim 10, further comprising one or more expansion elements extending between the distal support element and the proximal support element, and extendable when the tissue traction device is positioned adjacent target tissue to define a working area in which an instrument may be advanced unobstructed by any of the expansion elements.

16. A method of moving a region of target tissue away from a treatment site, said method comprising:
deploying a tissue traction device having a proximal support element, a distal support element, and a grasping rail adjacent the treatment site with the grasping rail spaced apart and across from the treatment site;
engaging the grasping rail with the target tissue; and
moving the grasping rail away from the treatment site to move the grasped target tissue away from the treatment site.

17. The method of claim 16, further comprising performing a procedure on the target tissue.

18. The method of claim 17, further comprising continuously applying force, via the grasping rail, on the grasped target tissue in a direction away from the treatment site to maintain the target tissue taut for performing the procedure thereon.

19. The method of claim 17, further comprising extending one or more expansion elements of the tissue traction device against tissue around the treatment site to form a working area for performing the procedure on the target tissue.

20. The method of claim 16, further comprising extending one or more expansion elements of the tissue traction device against tissue around the treatment site to hold the tissue of the treatment site taut.
